(19) United States
(12) Patent Application Publication
(10) Pub. No.: US 2007/0106122 A1
(43) Pub. Date: May 10, 2007

(54) INTUBATION ASSISTANCE APPARATUS

(75) Inventors: Hidetaka YOKOTA, Tokyo (JP); Yukio TANIGUCHI, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/552,235

(22) Filed: Oct. 24, 2006

(30) Foreign Application Priority Data
Oct. 24, 2005 (JP) P2005-309152
Jul. 25, 2006 (JP) P2006-202369

(51) Int. Cl. A61B 1/267; A61B 1/04

(52) U.S. Cl. 600/188; 600/131; 600/120; 600/178

(57) ABSTRACT

An intubation assistance apparatus includes a main body; an insertion instrument having an elongated insertion section for insertion into a trachea or its vicinity of a patient from a mouth cavity of the patient; and an imaging device for acquiring an image of an observation site at a distal end portion of the insertion instrument as an electronic image. The intubation assistance apparatus is adapted to be used in an assembled state that the main body, the insertion instrument and the imaging means are assembled together. The main body includes a display having a rectangular screen for displaying the electronic image taken by the imaging device in the assembled state. The display is rotatably provided on the proximal end portion of the main body so as to be rotatable between a first position where the display is close to the main body and a second position where the display is far away from the main body, in which the center of the screen is located on the central axis of the insertion instrument regardless of the rotation angle of the display when the display is viewed from the side of the screen in the assembled state.

### BACKGROUND OF THE INVENTION

1. Field of the Invention

The present invention relates to an intubation assistance apparatus for use in inserting a distal end of an intubation tube into a target site such as a trachea of a patient.

2. Description of the Prior Art

It is sometimes necessary to practice artificial respiration, as a first-aid lifesaving treatment for a patient who is suffering from unconsciousness caused by an accident or the like. Although the artificial respiration may be practiced without having to use any instrument or apparatus, it is often the case that a respirator is used for that purpose.

In the event that a respirator is utilized to conduct artificial respiration, an intubation tube whose proximal end is connected to the respirator is inserted into the trachea of a patient to supply air into the trachea from the respirator via the tube.

In the meantime, if a patient loses consciousness, the root of a tongue is retracted to thereby block up a respiratory tract (air passage) because of the relaxation of muscles of the pharynx and the larynx and/or the gravity caused loosening of a lower jaw.

Therefore, in the case where the afore-mentioned intubation tube is to be inserted into the trachea or a target area, it is essential to first open the blocked respiratory tract and secure an air passage by pulling up the tongue.

As an instrument for use in securing the air passage, there is known what is called an oral airway (see, e.g., US-A 2005-0150500).

Such an oral airway is an elongated member with a curved distal end and can be inserted through the mouth of a patient who has lost consciousness, whereby an appropriate portion on the side of the distal end comes into contact with and lifts up a tongue root portion of the patient, thus securing the air passage.

As described in the above-mentioned prior art, the oral airway (intubation assistance instrument) is normally used in a state that the proximal end of the intubation assistance instrument is coupled to a main body of an intubation assistance apparatus (hereinafter, this state will be referred to as “coupling state”). The intubation assistance apparatus includes an imaging means (image pick-up device) for taking an image of an observation site at a distal end portion of the intubation assistance instrument as an electronic image, and a display for displaying the electronic image taken by the imaging means.

The above-described intubation assistance apparatus in the coupling state is configured so that the display is positioned on the left side of the intubation assistance instrument at the time when the intubation assistance apparatus is used in a state shown in FIG. 1 of the prior art. Therefore, the center of the screen of the display is dislocated from the center line of the intubation assistance instrument on the left side thereof.

When such an intubation assistance apparatus is used, an operator inserts the intubation assistance instrument into the trachea of a patient through the mouth of the patient with monitoring the display. Therefore, in the prior art intubation assistance apparatus, the operator is likely to operate the apparatus with undesirable recognition that the center of the screen of the display is aligned with the center line of the intubation assistance instrument. For this reason, in the prior art intubation assistance apparatus, there is a problem in that an operator excessively moves the intubation assistance instrument with respect to the observation site due to the dislocation between the center of the screen and the center line of the intubation assistance instrument, thus resulting in the case that the manipulability of the apparatus when the intubation assistance instrument is inserted into the trachea of a patient is lowered.

### SUMMARY OF THE INVENTION

It is therefore an object of the present invention to provide an intubation assistance apparatus having a superior manipulability when an intubation assistance instrument is inserted into an air passage of a patient.

In order to achieve this object, the present invention is directed to an intubation assistance apparatus which includes a main body having a proximal end portion; an insertion instrument having an elongated insertion section for insertion into a trachea or its vicinity of a patient from a mouth cavity of the patient, the insertion instrument having a central axis; and imaging means for taking an image of an observation site at a distal end portion of the insertion instrument as an electronic image, wherein the intubation assistance apparatus is adapted to be used in an assembled state where the main body, the insertion instrument and the imaging means are assembled together. The main body includes a display having a screen for displaying the electronic image taken by the imaging means in the assembled state, and the display is arranged on the proximal end portion of the main body so that the center of the screen is located on the central axis of the insertion instrument when the display is viewed from the side of the screen in the assembled state.

The intubation assistance apparatus of the present invention having the above structures can exhibit superior manipulability when the insertion instrument is inserted into the trachea of a patient.

In the intubation assistance apparatus according to the present invention described above, it is preferred that the screen of the display has a rectangular shape having a center line which is in parallel with the long side of the rectangular shape, and wherein the display is arranged on the proximal end portion of the main body so that the center line is located on the central axis of the insertion instrument when the display is viewed from the side of the screen in the assembled state.

This makes it possible to exhibit more superior manipulability when the insertion instrument is inserted into the trachea of a patient.

Further, in the intubation assistance apparatus according to the present invention described above, it is preferred that the screen is inclined with respect to the central axis of the insertion instrument.

According to this configuration, though depending on the posture of the operator during the insertion operation, it is possible to direct the display to the operator during the insertion operation so that the display can be monitored easily.

Furthermore, in the intubation assistance apparatus according to the present invention described above, it is preferred that the display is rotatably provided on the proximal end portion of the main body so as to be movable between a first position where the display is close to the main body and a second position where the display is far away from the main body, and wherein the center of the screen is located on the central axis of the insertion instrument regardless of the rotation angle of the display when the display is viewed from the side of the screen in the assembled state.

This makes it possible for an operator to carry out the intubation operation reliably irrespective of the positional relationship between the operator and the patient when inserting the insertion instrument into the trachea of the patient.

Moreover, in the intubation assistance apparatus according to the present invention described above, it is preferred that the screen of the display has a rectangular shape having a center line which is in parallel with the long side of the rectangular shape, and wherein the display is arranged on the proximal end portion of the main body so that the center line is located on the central axis of the insertion instrument regardless of the rotation angle of the display when the display is viewed from the side of the screen in the assembled state.

This also makes it possible for an operator to carry out the intubation operation reliably irrespective of the positional relationship between the operator and the patient when inserting the insertion instrument into the trachea of the patient.

In the above modifications, it is preferred that the screen is inclined with respect to the central axis of the insertion instrument when the display is in the first position.

According to this configuration, though depending on the posture of the operator during the insertion operation, it is possible to direct or face the display to the operator during the insertion operation so that the display can be monitored easily.

In the intubation assistance apparatus according to the present invention, it is preferred that the main body includes a grip section which is to be grasped by an operator when the intubation assistance apparatus is used.

According to this configuration, an operator can grasp the intubation assistance apparatus in the assembled state easily and reliably, thus exhibiting superior manipulability when inserting the insertion instrument into the trachea of a patient.

In the modification described above, it is preferred that the grip section has an outer profile of a column-like shape, and the central axis of the grip section is substantially aligned with the central axis of the insertion instrument.

According to this configuration, since the hand of the operator who grasps the grip section is positioned near the screen of the display, it is possible to manipulate the insertion instrument accurately.

Further, in the intubation assistance apparatus according to the present invention, it is also preferred that the insertion instrument has a straight portion extending linearly and a curved portion continuously extending from the distal end of the straight portion and having a curved form.

According to this configuration, the insertion operation of the insertion instrument into the mouth of a patient can be carried out easily.

In the modification described above, it is preferred that the distal end of the curved portion is directed to a direction which is substantially in parallel with a normal line of the screen in the assembled state.

This also makes it possible to exhibit more superior manipulability when the insertion instrument is inserted into the trachea of a patient.

Furthermore, in the intubation assistance apparatus according to the present invention, it is also preferred that the insertion instrument has guide means for removably holding an intubation tube and for leading the intubation tube to the trachea or its vicinity of the patient.

This makes it possible to lead the intubation tube to the target site such as the trachea of the patient reliably.

Moreover, in the intubation assistance apparatus according to the present invention, it is also preferred that the imaging means includes an image pick-up device provided in the main body and leading means provided in the insertion instrument for leading an image of an object to be taken to the image pick-up device.

This makes it possible to take an electronic image of the observation site where the distal end of the insertion instrument is positioned, and the thus obtained electronic image can be monitored by the screen.

These and other objects, structures and results of the present invention will be apparent more clearly when the following detailed description of the preferred embodiments is considered taken in conjunction with the appended drawings.

### BRIEF DESCRIPTION OF THE DRAWINGS

FIG. 1 is a perspective view which shows a first embodiment of an intubation assistance apparatus according to the present invention.

FIG. 2 is another perspective view which shows the first embodiment of an intubation assistance apparatus according to the present invention.

FIG. 3 is an illustration which shows the intubation assistance apparatus shown in FIG. 1 viewed from the direction indicated by the arrow A in FIG. 1.

FIG. 4 is a cross-sectional view taken along line B-B in FIG. 1.

FIG. 5 is a perspective view which shows a main body of the intubation assistance instrument of the intubation assistance apparatus shown in FIGS. 1 and 2.

FIG. 6 is a perspective view which shows an insertion instrument of the intubation assistance instrument of the intubation assistance apparatus shown in FIGS. 1 and 2.

FIG. 7 is another perspective view which shows an insertion instrument of the intubation assistance instrument of the intubation assistance apparatus shown in FIGS. 1 and 2.

FIG. 8 is a perspective view of an intubation tube which is used in combination with the intubation assistance apparatus shown in FIGS. 1 and 2.

FIG. 9 is a perspective view which shows a second embodiment of an intubation assistance apparatus according to the present invention.

FIG. 10 is a perspective view of a main body of intubation assistance apparatus shown in FIG. 9.

### DETAILED DESCRIPTION OF THE PREFERRED EMBODIMENTS

Hereinbelow, preferred embodiments of an intubation assistance apparatus according to the present invention will be described in detail with reference to the accompanying drawings.

### First Embodiment

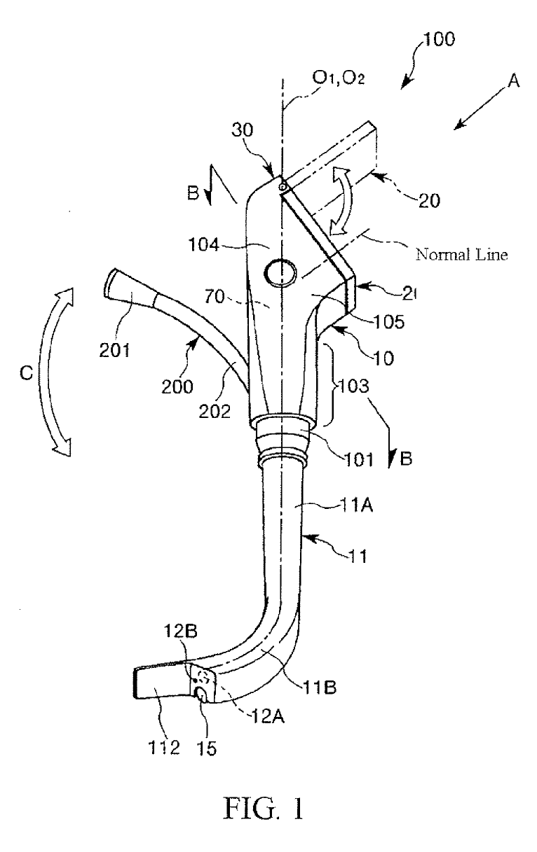

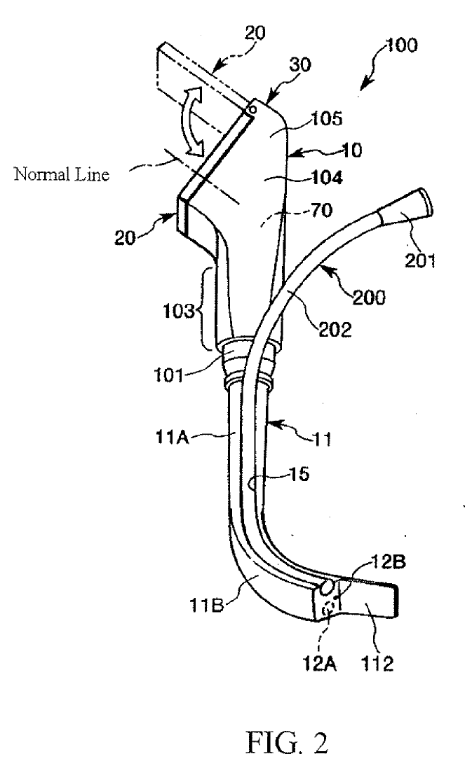

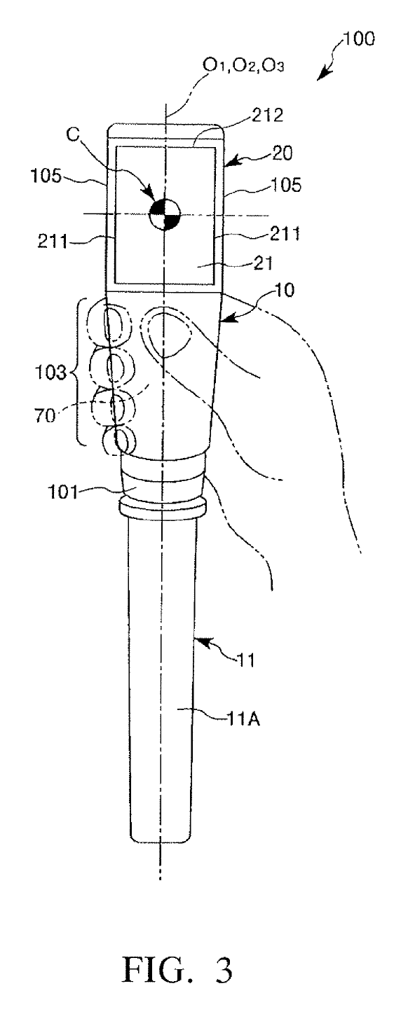

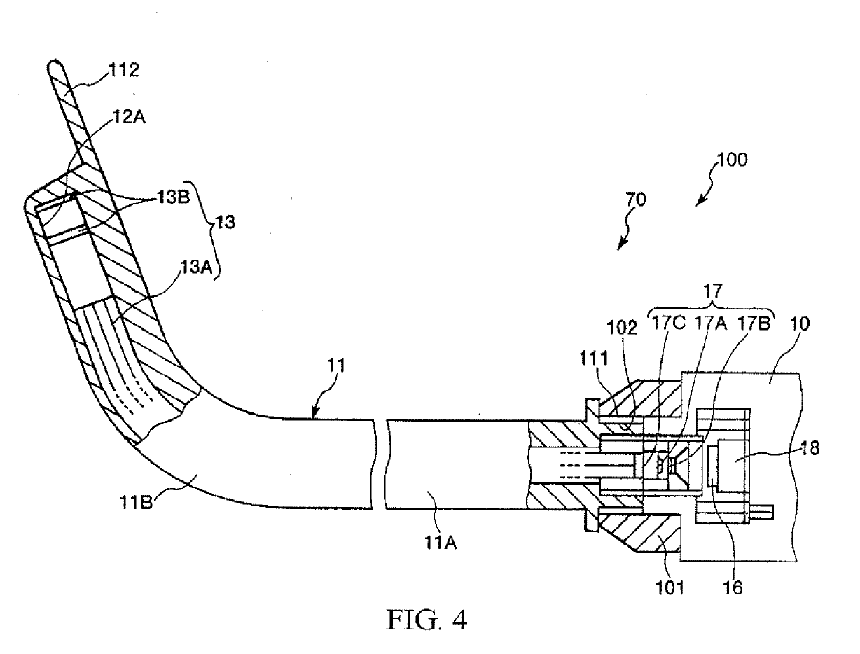

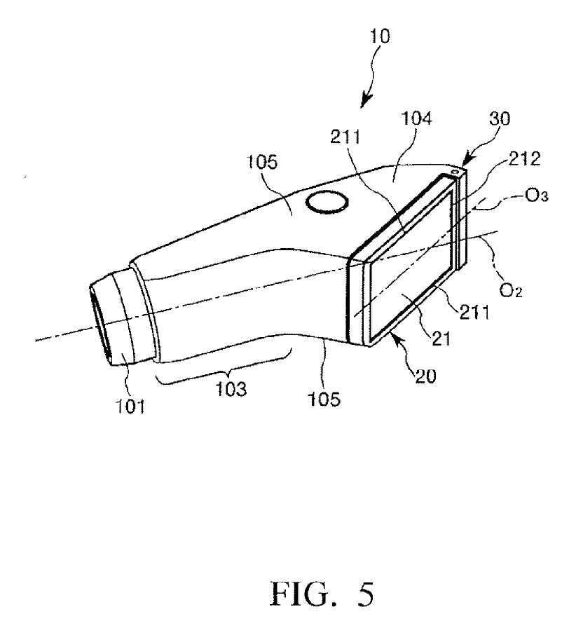

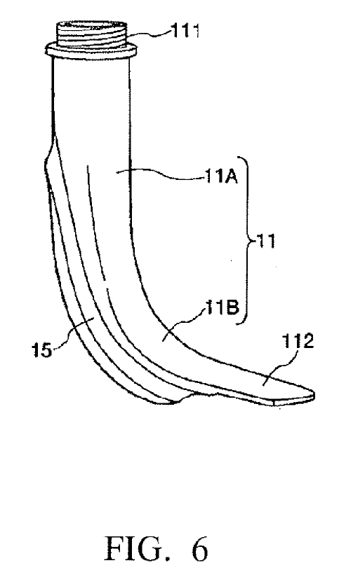

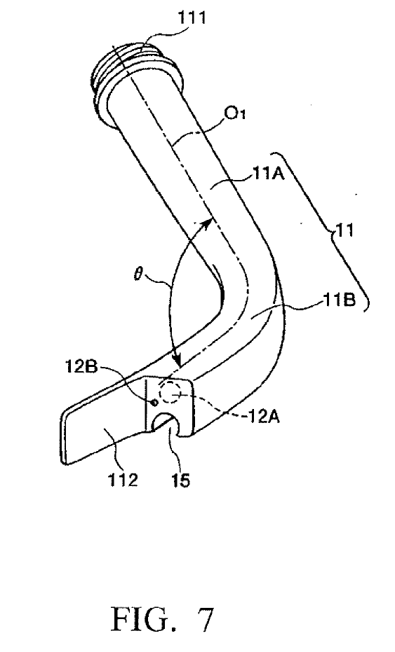

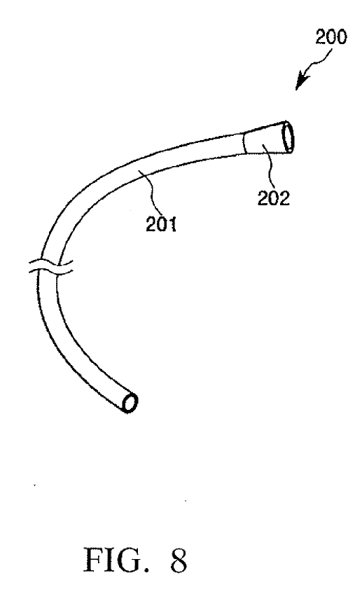

FIG. 1 is a perspective view which shows a first embodiment of an intubation assistance apparatus according to the present invention; FIG. 2 is another perspective view which shows the first embodiment of an intubation assistance apparatus according to the present invention; FIG. 3 is an illustration which shows the intubation assistance apparatus shown in FIG. 1 viewed from the direction indicated by the arrow A in FIG. 1; FIG. 4 is a cross-sectional view taken along line B-B in FIG. 1; FIG. 5 is a perspective view which shows a main body of the intubation assistance instrument of the intubation assistance apparatus shown in FIGS. 1 and 2; FIG. 6 is a perspective view which shows an insertion instrument of the intubation assistance instrument of the intubation assistance apparatus shown in FIGS. 1 and 2; FIG. 7 is another perspective view which shows an insertion instrument of the intubation assistance instrument of the intubation assistance apparatus shown in FIGS. 1 and 2; and FIG. 8 is a perspective view of an intubation tube which is used in combination with the intubation assistance apparatus shown in FIGS. 1 and 2. Note that in FIG. 3 an intubation tube is omitted. Further, in the following description, the upper side and the lower side in FIGS. 1 to 4 will be referred to as “proximal end” and “distal ends”, respectively, for the purpose of clarity.

The intubation assistance apparatus 100 shown in FIGS. 1 and 2 includes a main body 10, an insertion instrument 11 and an image taking means 70, and the intubation assistance apparatus 100 is used in a state that these components are assembled together.

As will be described later, the intubation assistance apparatus 100 is used in combination with an intubation tube 200 which is to be inserted into the trachea of a patient through the mouth (mouth cavity) thereof. In this regard, please note that although the main body 10 and the insertion instrument 11 are separate components in the followings embodiments, the main body 10 and the insertion instrument 11 may be formed into an integral body.

Hereinbelow, detailed descriptions will be made with regard to each of these components.

The insertion instrument 11 shown in FIGS. 6 and 7 is formed from an elongated member which constitutes an elongated insertion section, and it is used by being inserted into the trachea of a patient or its vicinity who has lost consciousness from the mouth (mouth cavity) of the patient. An air passage for the patient is secured by bringing an appropriate portion on the side of a distal end of the insertion instrument 11 into contact with the root of a tongue of the patient.

As illustrated in FIGS. 6 and 7, the insertion instrument 11 has a straight portion 11A and a curved portion 11B which is curved and extends from the distal end side of the straight portion 11A continuously. The outer circumferential surface of the straight portion is rounded to form a substantially column-like shape.

Further, the proximal end part of the straight portion 11A is formed with a male thread 111. The male thread 111 is adapted to be threaded with a female thread 102 formed in a coupling member 101 of the main body 10 (see FIG. 4). When the male thread 111 is threaded with the female thread 102, the insertion instrument 11 is mounted to the main body to provide the intubation assistance apparatus 100 in the assembled state.

Further, by disengaging the male thread 111 of the straight portion 11A from the female thread 102 of the main body 10, the insertion instrument 11 is removed from the main body 10. Therefore, by removing the insertion instrument 11 which has been used for a patient from the main body 10, it is possible to attach a new insertion instrument 11 to the main body 10.

As described above, the curved portion 11B extends from the distal end side of the straight portion 11A in a continuous manner. The curved portion 11B is curved so that the distal end thereof is directed to a direction inclined with respect to the straight portion 11A. The inclined angle θ (see FIG. 7) is not particularly limited, but it is preferably in the range of 70 to 140°, and more preferably in the range of 90 to 120°.

By constructing the insertion instrument 11 from the straight portion 11A and the curved portion 11B so that the entire shape thereof forms an L-shape, the insertion instrument 11 can be inserted into the mouth of a patient easily to thereby secure an air passage of the patient reliably.

Further, on the distal end of the curved portion 11B, a plate-shaped protruding portion (tongue piece) 112 is integrally formed. The protruding portion 112 is used for pushing up or lifting up the epiglottis or the root of tongue of the patient when securing the air passage of the patient.

As shown in FIG. 6, on the side portion of the insertion instrument 11, a groove (guide means) 15 is formed from the midway of the straight portion 11A to the distal end portion of the curved portion 11B along the central axis O₁ of the insertion instrument 11. This groove 15 has a function of guiding or leading an intubation tube 200 which is inserted from the mouth of a patient to the trachea of the patient after the air passage thereof has been secured by the insertion instrument 11.

In more details, the intubation tube 200 is inserted into the groove 15 as shown in FIG. 2 after the air passage of the patient has been secured, and then advanced toward the distal end of the insertion instrument 11. At this time, the intubation tube 200 is slidably advanced within the groove 15 with being guided by the inner wall of the groove. Then, the distal end of the intubation tube 200 protrudes from the distal end of the insertion instrument 11 and further advanced toward the rima glottidis or its vicinity of the patient which is located back of larynx thereof.

In this regard, it is to be noted that the cross-section of the groove 15 shown in the drawings has a substantially C-shape, but the cross-sectional shape there of is not limited thereto and it may be formed into a U-shape.

Further, it is preferred that the width (maximum width) of the groove 15 is set to be slightly larger than the outer diameter of the intubation tube 200. This makes it possible that the intubation tube 200 is stably held within the groove 15, that is, the intubation tube 200 can be smoothly advanced within the groove 15.

As shown in FIGS. 4 and 7, a first internal bore 12A is formed inside the insertion instrument 11 along the central axis O₁ of the insertion instrument 11. The first internal bore 12A has a substantially circular cross-section.

As shown in FIG. 4, the first internal bore 12A is opened at the proximal end of the insertion instrument 11 (that is, the straight portion 11A), and closed at the distal end of the insertion instrument 11 (that is, the curved portion 11B). In this regard, it is to be noted that a blockage portion for closing the distal end of the internal bore 12A is formed into a substantially transparent portion.

Inside the first internal bore 12A, there are provided a bundle of optical fibers 13A which constitutes a part of the image taking means and an objective lens 13B which is located in front of the bundle of optical fibers 13A.

Further, a second internal bore 12B having a diameter smaller than the diameter of the first internal bore 12A is also formed inside the insertion instrument 11. The second internal bore 12B is formed along the central axis O₁ of the insertion instrument 11 in the same manner as the first internal bore 11A. In other words, the second internal bore 12B is arranged in parallel with the first internal bore 12A.

Both the ends of the second internal bore 12 are opened at the proximal and distal ends of the insertion instrument 11, respectively, that is, the second internal bore 12B is formed into a through hole. By using such a second internal bore 12B, it is possible to aspirate substances inside the air passage, for instance, by means of a suction means such as a pump (not shown in the drawings) connected to the intubation assistance apparatus 100 in the assembled state.

The constituent material of the insertion instrument 11 is not particularly limited, but various polymer materials such as polycarbonate and the like are preferably used.

As shown in FIGS. 1 to 3 and 5, the main body 10 includes a grip section 103, a display 20, a coupling portion 104 for coupling the grip section 103 to the display 20, and a coupling member 101.

The grip section 103 is a portion of the intubation assistance apparatus 100 which is to be grasped by an operator with his one or both hands when the intubation assistance apparatus 100 in the assembled state is actually used (see FIG. 3). Note that FIG. 3 shows a state that the grip section 103 is grasped by an operator with his one hand.

The outer profile of the grip section 103 has a roughly column-like shape. Due to this shape, it is possible to grasp the grip section 103 easily and reliably, whereby enabling the insertion instrument 11 to be inserted into the trachea of a patient easily and reliably by manipulating the intubation assistance apparatus 100 in the assembled state.

On the distal end portion of the grip section 103, the coupling member 101 is mounted so that the coupling member 101 is rotatable about the central axis O₂ of the main body 10 (the grip section 103). The coupling member 101 is formed from an annular member.

Further, as described above, the female thread 102 is formed on the inner circumferential surface of the coupling member 101. As shown in FIG. 4, the female thread 102 is adapted to be threaded with the male thread formed on the proximal end portion of the insertion instrument 11.

On the proximal end portion of the grip section 103, the coupling portion 104 is provided so as to extend from the grip section 103. The display 20 is provided on the coupling portion 104.

As shown in FIGS. 1 and 2, the display 20 is provided on the coupling portion 104, that is, on the proximal end portion of the main body 10.

The display 20 is formed into a plate-like shape having a rectangular outer peripheral shape, and includes a screen 21 for displaying an electronic image taken by the image taking means 70 in the assembled state of the intubation assistance apparatus 100.

The screen 21 is constructed from a liquid crystal device or an organic EL device or the like, and displays an image (electronic image) based on image light taken by the image taking means 70. The screen 21 also has a rectangular shape corresponding to the rectangular outer peripheral shape of the display 21.

The display 20 is rotatably supported on the main body 10 through a rotational mechanism 30 with one of the short sides 212 of the screen 21 being used as a rotation axis thereof. As a result, the display 20 is rotatable with respect to the main body 10 along a plane extending from the side surface 105 of the main body 10.

Specifically, the display 20 is rotatable between a first position where the display 20 makes contact with the main body 10 (this position of the display 20 is shown by a solid line in FIGS. 1 and 2) and a second position where the display 20 is far away from the main body 10 (this position of the display 20 is shown in a two-dot chain line).

The rotational mechanism 30 may be constructed so that the display 20 is stopped at each of the first and second positions. Alternatively, the rotational mechanism 30 may be constructed so that the display 20 can be stopped at any desired position between the first and second positions in a nonstep manner, or so that the display 20 can be stopped at one of the predetermined positions (e.g. one of five steps) between the first and second positions in a stepwise manner.

As described above, since the display 20 is rotatable with respect to the main body 10, it is possible to direct the display 20 to a desired direction despite the direction of the insertion instrument 11 in the assembled state of the intubation assistance apparatus 100. As a result, an operation for inserting the insertion instrument 11 into a patient (hereinafter, this operation will be simply referred to as “insertion operation”) can be carried out easily and reliably. Further, since the insertion operation can be carried out without bending the cervical region of a patient rearwardly, high safeness can be realized.

Further, normally, during the insertion operation of the intubation assistance instrument 11 into the trachea of a patient, the direction and posture of the intubation assistance instrument 11 is changed or rotated variously. According to the present embodiment, by rotating the display 20 to an appropriate angle during the insertion operation, it is possible to monitor the display 20 at an optimum angle.

As shown in FIG. 5, the intubation assistance apparatus 100 of the present invention is configured so that the screen 21 is inclined with respect to the central axis O₂ of the insertion instrument 11 even when the display 20 is in its first position. Therefore, though depending on the posture of the operator during the insertion operation, it is possible to direct the display 20 to the operator without rotating the display 20 during the insertion operation. Namely, the display 20 can be monitored easily during the insertion operation.

Next, a description will be made with regard to the image taking means 70.

The image taking means 70 shown in FIG. 4 is provided to take an image of an observation site where the distal end of the insertion instrument 11 is positioned as an electronic image. The image taking means 70 includes an image guide 13, an enlargement optical system 17, a CCD (image pick-up device), and a controller 18, and these components are arranged in this order along the longitudinal direction of the insertion instrument 11.

In the assembled state, the image guide 12 is arranged inside the first internal bore 12A. The image guide 13 is composed from a bundle of optical fibers 13A and an objective lens 13B provided in front of the bundle of optical fibers 13A.

The image guide 13 takes reflected light (image light) reflected from the observation site where the distal end of the insertion instrument 11 is positioned through the objective lens 13B, and the thus obtained image light (image of an object) is transmitted to the CCD through the bundle of optical fibers 13A. With this structure, when the air passage of the patient is secured by the insertion instrument 11, it is possible to acquire image light (electronic image) of the rima glottidis or its vicinity (that is, a region from the larynx to the rima glottidis) of the patient.

Namely, in this embodiment the image guide 13 constitutes means for leading image light of an observation site (image of an object) to the image pick-up device.

The bundle of optical fibers 13A is constructed by bundling a number of optical fibers made of quartz, multi-component glass, plastics or the like.

The CCD 16 is provided inside the main body 10. In the main body 10, the CCD 16 is disposed at a position facing the proximal end of the image guide in the assembled state of the intubation assistance apparatus 100, and an image is formed thereon based on the image light acquired by means of the image guide 13. Namely, an image of the observation site where the distal end of the insertion instrument 11 is positioned is formed by the CCD 16.

The enlargement optical system 17 is composed from magnifying lenses 17A and 17B and a diaphragm 17C, and the enlargement optical system 17 is arranged between the proximal end of the image guide 13 and the CCD 16. With this arrangement, an image based on the image light is formed on the CCD 16 with being enlarged. Note that the enlargement optical system that can be used in this embodiment is not limited to the optical system described above.

The controller 18 for displaying the electronic image on in the display 20 (on the screen 21) is also provided inside the main body 10. The screen 21 of the display 20 displays the electronic image formed by the CCD 16 under the control of the controller 11.

Examples of the image that is to be displayed on the screen 21 of the display 20 include an image showing the rima glottides of a patient in a state that the air passage of the patient is secured by the insertion instrument 11.

Next, a description will be made with regard to an intubation tube 200 that is used in combination with the intubation assistance apparatus 100.

As shown in FIG. 8, the intubation tube 200 is composed from a tube main body 201 and a connector 202 affixed to one end portion of the tube main body 201.

The tube main body 201 is made of an elastic material such as elastomer, rubber and the like. Further, the tube main body 201 has a substantially circular cross-section.

The connector 202 is used by being connected to a respirator when artificial respiration is carried out using the respirator. By using the intubation tube 200 having the above structure, it is possible to feed air into the trachea of a patient from the respirator with the state that the intubation tube 200 is being inserted into the trachea of the patient.

Hereinbelow, a description will be made with regard to the steps for assembling the intubation assistance apparatus 100.

In assembling the intubation assistance apparatus 100, the proximal end of the image guide 13 (bundle of optical fibers 13A) is first connected to the main body 10.

Thereafter, the image guide 13 which has been connected to the main body 10 is inserted into the first internal bore 12A of the insertion instrument 11.

Next, the male thread 111 of the insertion instrument 11 is threaded with the female thread 102 of the main body 10. In this regard, it is to be noted that the method for coupling the insertion instrument 11 to the main body 10 is not limited to the method mentioned above, and other various methods can be employed. Examples of such methods include a ratchet mechanism method, a bayonet mounting method, a cam method, a locking claw method and a magnetic method.

In the intubation assistance apparatus 100 assembled as described above, the center C of the screen 21 is located on (coincided with) the central axis O₁ of the insertion instrument 11 and further the central axis O₃ of the screen 21 which is in parallel with the long side of the rectangular screen 21 is also located on the central axis O₁ of the insertion instrument 11 regardless of the rotation angle of the display 20, when the display 20 is viewed from the side of the screen 21.

For example, as shown in FIG. 3, in the case where the display 20 is in the first position, the center C of the screen 21 is located on the central axis O₁ of the insertion instrument 11 and further the central axis O₃ of the screen 21 is also located on the central axis O₁ of the insertion instrument 11, when the display 20 is viewed from the direction of the arrow A in FIG. 1.

Further, even in the case where the display 20 is in the second position, the center C of the screen 21 is located on the central axis O₁ of the insertion instrument 11 and further the central axis O₃ of the screen 21 is also located on the central axis O₁ of the insertion instrument 11, when the display 20 is viewed from the side of the screen 21.

Furthermore, even in the case where the display 20 is in the arbitral position between the first and second positions, the center C of the screen 21 is located on the central axis O₁ of the insertion instrument 11 and further the central axis O₃ of the screen 21 is also located on the central axis O₁ of the insertion instrument 11, when the display 20 is viewed from the side of the screen 21.

As described above, since the display 20 is arranged so that the center C of the screen 21 is located on the central axis O₁ of the insertion instrument 11, the dislocation of the center C of the screen 21 from the central axis O₁ of the insertion instrument 11 which is involved by the prior art intubation assistance apparatus described above can be overcome. As a result, it is possible for an operator to manipulate the insertion instrument 11 (that is, the intubation assistance apparatus 100) on the basis of the visual recognition of the operator for the image displayed on the screen 21 during the insertion operation.

Further, in this embodiment, when the insertion instrument 11 (that is, the intubation assistance apparatus 100) is manipulated, the insertion instrument 11 is pivotally displaced with the grip section 103 grasped by an operator being a center of the pivotal motion (which is shown by the arrow C in FIG. 1). As a result, the distal end of the insertion instrument 11 is also pivotally swung in a substantially predetermined direction. Therefore, the image displayed on the screen 21 moves roughly along the central axis O₁ of the insertion instrument 11 (that is, along top and bottom directions in FIG. 3). In this embodiment, since the display 20 is arranged so that the central axis O₃ of the screen 21 is located on the central axis O₁ of the insertion instrument 11, it is possible to widen a region (that is, an observation region or a field of view) in the direction (that is, top and bottom directions in FIG. 3) along which the image displayed on the screen 21 can move rather than a region in the direction (that is, left and right directions in FIG. 3) along which the image displayed on the screen 21 can hardly move. As a result, the manipulability of the intubation assistance apparatus 100 during the insertion operation can be made excellent.

Further, as shown in FIGS. 1 and 2, in the assembled state of the intubation assistance apparatus 100, the distal end portion of the insertion instrument 11 (that is, the distal end portion of the curved portion 11B) is directed to a direction which is substantially in parallel with a normal line of the screen 21 of the display 20 at the first position thereof.

With this structure, since the direction in which the observation site can be seen through the objective lens 13B is substantially the same as the direction through which the operator monitors the screen 21 of the display 20, the operator can manipulate the intubation assistance apparatus 100 based on the image displayed on the screen 21 of the display 20. Further, according to this embodiment, the intubation assistance apparatus 100 in the assembled state has a roughly straight configuration, and therefore when the intubation assistance apparatus 100 is placed on a table (not shown in the drawings), the intubation assistance apparatus 100 can be placed on the table in a stable manner.

Furthermore, the intubation assistance apparatus 100 may be provided with a direction regulating means for regulating the coupling direction of the insertion instrument 11 with respect to the main body 10 so that the distal end portion of the insertion instrument 11 in the assembled state is directed to a direction which is substantially opposite to the direction that the screen 21 of the display 20 faces at the first position thereof. The structure of such regulating means is not limited to a specific one, but as one examples thereof, it is possible to mention a structure in which a rib-shaped convex portion (guide pin) is formed on the distal end surface of the grip section 103 and a concave portion (guide hole) is formed on the distal end surface of the insertion instrument 11 so that the convex portion is engaged with the concave portion in the assembled state of the intubation assistance apparatus 100.

As shown in FIGS. 1 and 3, in the assembled state of the intubation assistance apparatus 100, the central axis O₂ of the grip section 103 of the main body 10 is located on the central axis O₁ of the insertion instrument 11. With this structure, since the hand of the operator who grasps the grip section 103 is positioned near the lower part of the display 21, it is possible to manipulate the insertion instrument 11 accurately.

Next, a description will be made with regard to one exemplary use (operation) of the intubation assistance apparatus 100 in the assembled state.

The intubation assistance apparatus 100 is used in such an instance that a patient has lost consciousness and a need exists to insert the intubation tube 200 into the trachea of the patient.

[1] First, the intubation assistance apparatus 100 is assembled in preparation for insertion of the intubation tube 200, and then confirmation is made as to whether or not the LED for illumination (not shown in the drawings) and the liquid crystal image are properly operated.

[2] Next, the insertion instrument 11 of the intubation assistance instrument 100 is inserted (pushed) into the trachea of the patient through the mouth of the patient. More specifically, the insertion instrument 11 is inserted into the mouth of the patient, while allowing the inner side of the curved portion 11B of the insertion instrument 11 to extend along the root of the tongue. As a result, an appropriate area on the distal end side of the insertion instrument 11 is brought into contact with the tongue root portion of the patient, thus securing an air passage of the patient.

As described above, in the intubation assistance instrument 100 of the present invention, since the display 20 is arranged so that the center C of the screen 21 is located on the central axis O₁ of the insertion instrument 11, it is possible for an operator to manipulate the insertion instrument accurately with monitoring the screen 21 of the display 20, and thus it is possible to insert the insertion instrument 11 into the trachea of the patient reliably.

Further, as described above, the display is provided on the main body 10 in a rotatable manner. Although the direction and posture of the insertion instrument 11 is changed or rotated variously during the insertion operation of the insertion instrument 11 into the trachea of a patient, it is possible to monitor the display 20 at an optimum angle by rotating the display 20 to an appropriate angle during the insertion operation.

Furthermore, as described above, the image displayed on the screen 21 moves roughly along the central axis O₁ of the insertion instrument 11 during the insertion operation of the insertion instrument 11. However, since the display 20 is arranged so that the central axis O₃ is located on the central axis O₁ of the insertion instrument 11, it is possible to monitor the image being displaced in the screen 21 over a relatively wide region along the moving direction of the image. This also makes it possible to carry out the insertion operation of the insertion instrument 11 easily.

[3] Once the air passage is secured by the distal end of the insertion instrument 11, an image of the rima glottidis and its vicinity of the patient (that is, the observation site) is taken

[4] Then, data of the image of the object obtained in the previous step is sent to the display 20 through the controller 18, and an electronic image of the rima glottidis and its vicinity of the patient is displayed on the screen 21 of the display 20.

[5] Then, the intubation tube 200 is inserted into the groove 15 from the proximal end portion of the insertion instrument 11 and continues to be pushed forward. In this process, the intubation tube 200 is guided by the groove 15 and advanced or moved forward along the groove 15 to protrude out of the groove 15 at the distal end of the insertion instrument 11. Observing the image displayed on the screen 21 (including the image of the distal end portion of the intubation tube 200), the operator inserts the distal end portion of the intubation tube 200, which protrudes from the groove 15, into the rima glottidis so that it can reach the trachea.

[6] Under the state that the intubation tube 200 remains inserted into the trachea, the intubation tube 200 is deformed and detached from the groove 15. While maintaining this condition, the insertion instrument 11 is removed or taken out from the mouth of the patient so that only the intubation tube 200 remains being inserted into the mouth of the patient.

In the manner as described above, the intubation tube 200 can be intubated into the trachea of the patient.

In this regard, it is to be noted that in general the proximal end of the intubation tube 200 is connected to an artificial respirator, and air is fed into the trachea of a patient from the respirator through the intubation tube 200 which is inserted into the trachea of the patient through the rima glottidis thereof.

### Second Embodiment

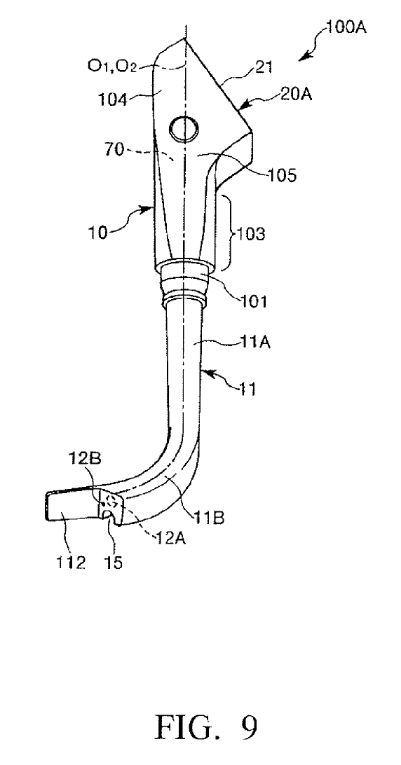

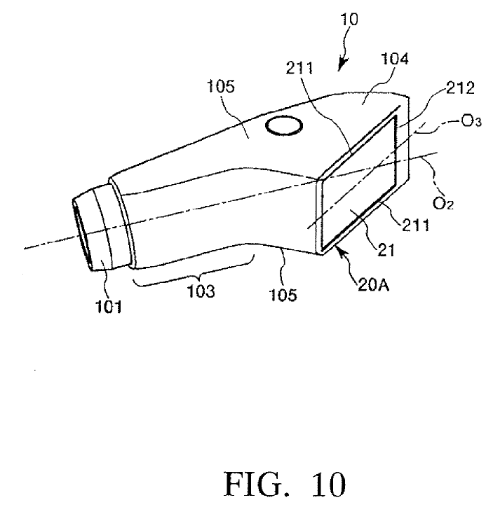

FIG. 9 is a perspective view which shows a second embodiment of an intubation assistance apparatus according to the present invention, and FIG. 10 is a perspective view of a main body of intubation assistance apparatus shown in FIG. 9.

Hereinbelow, the second embodiment of the intubation assistance apparatus according to the present invention will be described based on these drawings, but the description will focus on the different portions from the first embodiment and a description on the common portions is omitted.

The structure of the second embodiment is the same as that of the first embodiment excepting that the arrangement of the display with respect to the main body is different that of the first embodiment.

As shown in FIGS. 9 and 10, in the intubation assistance apparatus 100A of the second embodiment, the display 20A is fixedly mounted on the proximal end portion of the main body 10. The position in which the display 20A is fixedly mounted may be the same position as the first position of the display 20 of the first embodiment.

Therefore, in substantially the same manner as the fist embodiment, the center C of the screen 21 is located on the central axis O₁ of the insertion instrument 11 and further the central axis O₃ of the screen 21 which is in parallel with the long side of the rectangular screen 21 is also located on the central axis O₁ of the insertion instrument 11, when the display 20A is viewed from the side of the screen 21 in the assembled state of the intubation assistance apparatus 100A.

As described above, since the display 20A is arranged so that the center C of the screen 21 is located on the central axis O₁ of the insertion instrument 11, the dislocation of the center C of the screen 21 from the central axis O₁ of the insertion instrument 11 which is involved by the prior art intubation assistance apparatus described above can be overcome due to the same reasons as the first embodiment. As a result, it is possible for an operator to manipulate the insertion instrument 11 (that is, the intubation assistance apparatus 100A) on the basis of the visual recognition of the operator for the image displayed on the screen 21 during the insertion operation. Further, it is also possible to prevent erroneous manipulation. For example, it is possible to prevent the insertion instrument 11 from being manipulated toward improper directions or from being moved excessively.

Further, as is the same with the first embodiment described above, when the insertion instrument 11 (that is, the intubation assistance apparatus 100A) is manipulated, the insertion instrument 11 is pivotally displaced with the grip section 103 grasped by an operator being a center of the pivotal motion. As a result, the distal end of the insertion instrument 11 is also pivotally swung in a substantially predetermined direction. Therefore, an image displayed on the screen 21 is displaced roughly along the central axis O₁ of the insertion instrument 11. In addition, since the display 20A is arranged so that the central axis O₃ of the screen 21 is located on the central axis O₁ of the insertion instrument 11, it is possible to widen a region (that is, an observation region or a field of view) in the direction along which the image displayed on the screen 21 can move rather than a region in the direction along which the image displayed on the screen 21 can hardly move. As a result, the manipulability of the intubation assistance apparatus 100A during the insertion operation can be made excellent.

Furthermore, the screen 21 of the display 21A is inclined with respect to the central axis O₁ of the insertion instrument 11. Therefore, though depending on the posture of the operator during the insertion operation, it is possible to direct the display 20A to the operator during the insertion operation. Namely, the display 20A can be monitored easily during the insertion operation.

While the intubation assistance apparatus of the present invention has been described hereinabove in respect of the illustrated embodiments, this is not intended to limit the scope of the present invention. Instead, each component or element of the intubation assistance apparatus may be replaced with other one that exhibits the same or similar function. Furthermore, other arbitrary components than disclosed above may be added thereto.

Finally, it is also to be understood that the present disclosure relates to subject matters contained in Japanese Patent Applications No. 2005-309152 filed on Oct. 24, 2005 and 2006-202369 filed on Jul. 25, 2006 which are expressly incorporated herein by reference in their entireties.

What is claimed is:

1. An intubation assistance apparatus, comprising:
a main body having a proximal end portion;
an insertion instrument having an elongated insertion section for insertion into a trachea or its vicinity of a patient from a mouth cavity of the patient, the insertion instrument having a central axis; and
imaging means for taking an image of an observation site at a distal end portion of the insertion instrument as an electronic image,
wherein the intubation assistance apparatus is adapted to be used in an assembled state where the main body, the insertion instrument and the imaging means are assembled together, and
wherein the main body includes a display having a screen for displaying the electronic image taken by the imaging means in the assembled state, and the display is arranged on the proximal end portion of the main body so that the center of the screen is located on the central axis of the insertion instrument when the display is viewed from the side of the screen in the assembled state.

2. The intubation assistance apparatus as claimed in claim 1, wherein the screen of the display has a rectangular shape having a center line which is in parallel with the long side of the rectangular shape, and wherein the display is arranged on the proximal end portion of the main body so that the center line is located on the central axis of the insertion instrument when the display is viewed from the side of the screen in the assembled state.

3. The intubation assistance apparatus as claimed in claim 1, wherein the screen is inclined with respect to the central axis of the insertion instrument.

4. The intubation assistance apparatus as claimed in claim 1, wherein the display is rotatably provided on the proximal end portion of the main body so as to be movable between a first position where the display is close to the main body and a second position where the display is far away from the main body, and wherein the center of the screen is located on the central axis of the insertion instrument regardless of the rotation angle of the display when the display is viewed from the side of the screen in the assembled state.

5. The intubation assistance apparatus as claimed in claim 4, wherein the screen of the display has a rectangular shape having a center line which is in parallel with the long side of the rectangular shape, and wherein the display is arranged on the proximal end portion of the main body so that the center line is located on the central axis of the insertion instrument regardless of the rotation angle of the display when the display is viewed from the side of the screen in the assembled state.

6. The intubation assistance apparatus as claimed in claim 4, wherein the screen is inclined with respect to the central axis of the insertion instrument when the display is in the first position.

7. The intubation assistance apparatus as claimed in claim 1, wherein the main body includes a grip section which is to be grasped by an operator when the intubation assistance apparatus is used.

8. The intubation assistance apparatus as claimed in claim 7, wherein the grip section has an outer profile of a column-like shape, and the central axis of the grip section is substantially aligned with the central axis of the insertion instrument.

9. The intubation assistance apparatus as claimed in claim 1, wherein the insertion instrument has a straight portion extending linearly and a curved portion continuously extending from the distal end of the straight portion and having a curved form.

10. The intubation assistance apparatus as claimed in claim 9, wherein the distal end of the curved portion is directed to a direction which is substantially in parallel with a normal line of the screen in the assembled state.

11. The intubation assistance apparatus as claimed in claim 9, wherein the insertion instrument has guide means for removably holding an intubation tube and for leading the intubation tube to the trachea or its vicinity of the patient.

12. The intubation assistance apparatus as claimed in claim 9, wherein the imaging means includes an image pick-up device provided in the main body and leading means provided in the insertion instrument for leading an image of an object to be taken to the image pick-up device.
